(19) United States
(12) Patent Application Publication
(10) Pub. No.: US 2003/0108523 A1
(43) Pub. Date: Jun. 12, 2003

(54) Cancer treatment system

(76) Inventors: James M. Lipton, Woodland Hills, CA (US); Anna P. Catania, Milan (IT)

Correspondence Address: PERKINS COIE LLP, POST OFFICE BOX 1208, SEATTLE, WA, 98111-1208, US

(21) Appl. No.: 10/235,682

(22) Filed: Sep. 5, 2002

Related U.S. Application Data
(60) Provisional application No. 60/317,514, filed on Sep. 5, 2001.

(51) Int. Cl. A61K 48/00; A61K 38/24

(52) U.S. Cl. 424/93.2; 514/44; 514/12

(57) ABSTRACT

An invention is disclosed for combating cancer. In vitro studies have shown that α-MSH inhibits the proliferation of various mesothelioma cell lines. The invention is directed to a system and method for combating cancer and, in a specific embodiment, mesothelioma. Use of a therapeutic composition containing α-MSH and/or derivatives of α-MSH is disclosed for treatments including but not limited to parenteral administrations, direct targeting of cancer cells, gene therapy and local administrations using a cannula. Certain derivatives of α-MSH, NDP-α-MSH for example, are particularly effective in combating growth in mesothelioma cell lines.

### BACKGROUND

The field of the present invention relates to cancer treatment. Cancer is a group of many related diseases that are characterized by uncontrolled cell growth and division. Oftentimes, the cancerous cells are associated with genetic mutations affecting genes involved in cell-cycle regulation. Inside the body, these cells may grow and accumulate into tumors. They may also metastasize or spread to other parts of the body from where the tumor was originally formed. However, not all cancer cells metastasize. Some tumors are considered benign tumors in that they do not invade other parts of the body. On the other hand, metastasizing tumors are considered malignant.

One example of cancer is malignant mesothelioma (MM), which is uniformly fatal. Mesotheliomas are neoplasms of the serosal membranes found in body cavities such as the pleura, peritoneum, pericardium, tunica vaginae, testis, and ovarian epithelium. About eighty percent of mesotheliomas originate in the pleural space and they represent the most common primary tumor of the pleural cavity. (Pisani et al., Mayo Clin. Proc. 63: 1234-1244, 1988). Because of an aging population of asbestos-exposed individuals, incidence of MM is expected to rise over the next 20 years. (Pete J, Hodgson J T, Matthews F E, “Jones J R: Continuing increase in mesothelioma mortality in Britain,” Lancet 1995, 345:535-539).

Neither chemotherapy nor surgery has been shown to prolong survival of patients with MM. (Pass H I et al., Chest 116:455S-460S, 1999). Several properties of MM render it resistant to conventional therapies. For example, MM is usually diffuse rather than localized, affecting first the parietal and then the visceral pleurae. Further, MM has a propensity to infiltrate the underlying and neighboring structures, especially the lung, diaphragm, chest wall, and mediastinum. These features currently make complete surgical resection impossible.

Considering the unavailability of a complete surgical option, it has been suggested that the combination of extrapleural pneumonectomy, chemotherapy, and radiotherapy may be able to combat MM. This therapeutic regimen attracted much interest, but the subsequent results were disappointing. Id.

Hence, there exists a need for new therapeutic ways and drugs to inhibit proliferation of mesothelioma, and of cancer, in general.

### SUMMARY OF THE INVENTION

The present invention is directed to a system and method for combating cancer and, in a specific embodiment, mesothelioma. One aspect of this invention involves the use of a therapeutic composition comprising α-MSH (SYSMEHFRWGKPV (SEQ. ID. NO. 4)), and/or derivatives of α-MSH to combat cancer. Examples of α-MSH derivatives may include, but are not limited to, chemically-modified α-MSH, α-MSH dimers, truncated α-MSH such as KPV (SEQ. ID. NO. 1), HFRWGKPV (SEQ. ID. NO. 3), and chemically modified or dimer forms thereof.

In another aspect of the invention, α-MSH and/or its derivatives may be delivered, as a peptide therapeutic or as a gene therapy medicine, locally (preferably in the case of mesothelioma) or systemically. In another aspect of the invention, α-MSH or its derivatives may be linked or associated with a recognition molecule such as an antibody or a ligand that recognizes cancerous cells. The recognition molecule may function as a targeting molecule to specifically deliver α-MSH and/or its derivatives to the specific cancer cells.

### BRIEF DESCRIPTION OF THE DRAWINGS

FIG. 1 shows the percent inhibitory effect of NDP-α-MSH on mesothelioma cell proliferation.

FIG. 2 shows dose-dependent inhibition of mesothelioma cell proliferation by NDP-α-MSH.

FIG. 3 shows the immunoreactivity of a mesothelioma cell line to MC-1R, a melanocortin receptor.

FIG. 4 shows α-MSH inhibition of NF-κB activation in dividing cells.

FIG. 5 shows an example of a KPV homodimer for use with one embodiment of the present invention.

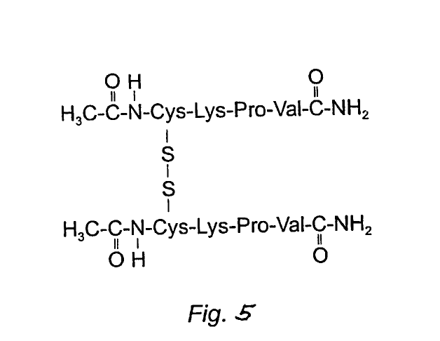

### GENERAL DESCRIPTION OF THE INVENTION

In this disclosure, the novel use of α-MSH and/or its derivatives for inhibiting proliferation of cancer cells is described. In vitro studies have shown that α-MSH, for example, inhibits the proliferation of various mesothelioma cell lines. The amount of inhibition varies from about 10% to 40% among different mesothelioma cells lines. Sensitivity of each cell line, however, was consistent across separate experiments suggesting that sensitivity to α-MSH is an intrinsic characteristic of each tumor cell line. Thus, α-MSH and/or its derivatives may be used as a therapeutic agent for combating mesothelioma, by itself or in combination with other treatment methods such as chemotherapy, radiotherapy, surgery, anti-angiogenic therapy, and any other suitable combination of treatments.

α-MSH is an ancient thirteen amino-acid peptide (SYSMEHFRWGKPV (SEQ. ID. NO. 4)) produced by post-translational processing of the larger precursor molecule, propiomelanocortin. It shares the 1-13 amino acid sequence with adrenocorticotropic hormone (“ACTH”), also derived from propiomelanocortin. α-MSH is known to be secreted by many cell types including pituitary cells, monocytes, melanocytes, and keratinocytes. It can be found in the skin of rats, in the human epidermis, or in the mucosal barrier of the gastrointestinal tract in intact and hypophysectomized rats. See e.g. Eberle, A. N., The Melanotrophins, Karger, Basel, Switzerland (1998); Lipton, J. M., et. al., “Anti-inflammatory Influence of the Neuroimmunornodulator α-MSH,” Immunol. Today 18, 140-145 (1997); Thody, A. J., et. al., “MSH Peptides are Present in Mammalian Skin,” Peptides 4, 813-815 (1983); Fox, J. A., et. al., “Immunoreactive α-Melanocyte Stimulating Hormone, Its Distribution in the Gastrointestinal Tract of Intact and Hypophysectomized Rats,” Life. Sci. 18, 2127-2132 (1981). These references, as well as all those used in this specification, are fully incorporated as if fully set forth herein.

α-MSH and its derivatives have been known to have potent antipyretic and anti-inflammatory properties, yet they have extremely low toxicity. They can reduce production of host cells' proinflammatory mediators in vitro, and can also reduce production of local and systemic reactions in animal models for inflammation. The “core” α-MSH sequence (4-10) (MEHFRWG, SEQ. ID. NO. 2), for example, has effects on learning and memory but little antipyretic and anti-inflammatory activity. In contrast, the active message sequence for the antipyretic and anti-inflammatory activities resides in the C-terminal amino-acid sequence of α-MSH, that is, lysine-proline-valine (“Lys-Pro-Val” or “KPV”) (SEQ. ID. NO. 1). This tripeptide has activities in vitro and in vivo that parallel those of the parent molecule.

The anti-inflammatory activity of α-MSH and/or its derivatives are disclosed in the following two patents and are hereby incorporated by reference: U.S. Pat. No. 5,028,592, issued on Jul. 2, 1991 to Lipton, J. M., entitled Antipyretic and Anti-inflammatory Lys Pro Val Compositions and Method of Use; U.S. Pat. No. 5,157,023, issued on Oct. 20, 1992 to Lipton, J. M., entitled Antipyretic and Anti-inflammatory Lys Pro Val Compositions and Method of Use; see also Catania, A., et. al., “(α-Melanocyte Stimulating Hormone in the Modulation of Host Reactions,” Endocr. Rev. 14, 564-576 (1993); Lipton, J. M., et al., “Anti-inflammatory Influence of the Neuroimmunomodulator of α-MSH,” Immunol. Today 18, 140-145 (1997); Rajora, N., et. al., “MSH Production Receptors and Influence on Neopterin, in a Human Monocyte/macrophage Cell Line,” J. Leukoc. Biol. 59, 248-253 (1996); Star, R. A., et. al., “Evidence of Autocrine Modulation of Macrophage Nitric Oxide Synthase by α-MSH,” Proc. Nat'l Acad. Sci. (USA) 92, 8015-8020 (1995); Lipton, J. M., et. al., “Anti-inflammatory Effects of the Neuropeptide α-MSH in Acute Chronic and Systemic inflammation,” Ann. N.Y. Acad. Sci. 741, 137-148 (1994); Fajora, N., et.al., “α-MSH Modulates Local and Circulating tumor Necrosis Factor in Experimental Brain Inflammation,” J. Neurosci, 17, 2181-2186 (1995); Richards, D. B., et. al., “Effect of α-MSH (11-13) (lysine-proline-valine) on Fever in the Rabbit,” Peptides 5, 815-817 (1984); Hiltz, M. E., et. al., “Anti-inflammatory Activity of a COOH-terminal Fragment of the Neuropeptide α-MSH,” FASEB J. 3, 2282-2284 (1989).

In addition to its anti-inflammatory and anti-pyretic function, α-MSH and/or its derivatives has also been shown to display anti-microbial or anti-infection activity. α-MSH and/or its derivatives have significant anti-infection uses, including, for example, use in reducing the viability of microbes, reducing the germination of yeast, killing microbes without reducing the killing of microbes by human neutrophils, for treating inflammation associated with microbial infection, increasing the accumulation of cAMP in microbes and inhibiting the replication and expression of viral pathogens. See PCT Publication WO 00/59527, published Oct. 12, 2000, and PCT Publication WO 00/56363, published 5 Sep. 28, 2000.

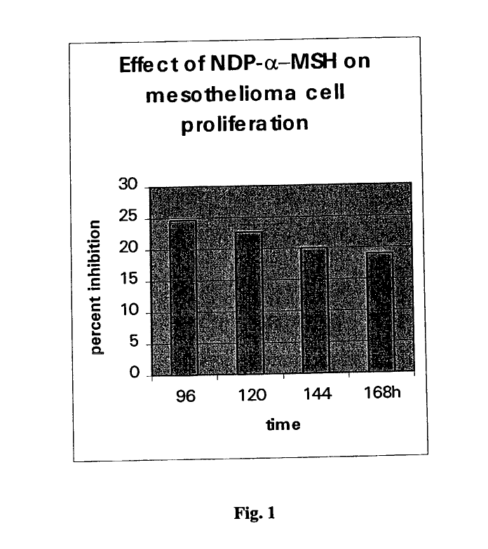

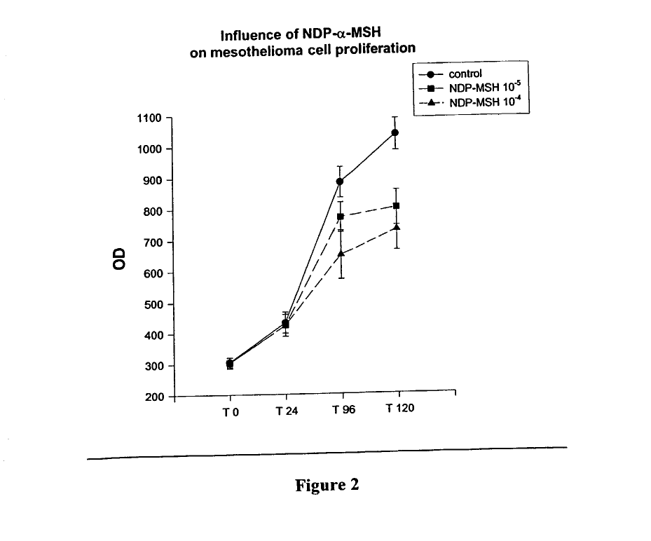

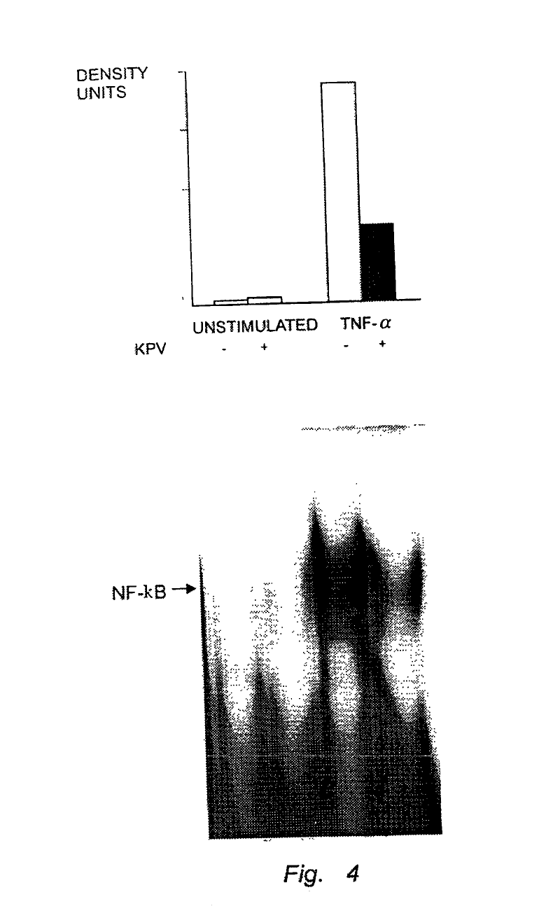

The finding that α-MSH and/or its derivatives exert inhibitory effects on the proliferation of mesothelioma cells represents a novel approach to combating cancer. It is currently believed that α-MSH and/or its derivatives inhibits proliferation of mesothelioma by affecting the NF-κB signal transduction pathway of the cells. In particular, NF-κB activation has been associated with tumorigenesis. As seen in FIG. 4, KPV, a derivative of α-MSH, inhibits the activation of NF-κB in dividing cells. Thus, it is believed that α-MSH and/or its derivatives inhibit proliferation of mesothelioma cells by inhibiting NF-κB. Since the activities of KPV, for example, and various derivatives of α-MSH parallel the activities of α-MSH, KPV, α-MSH, and its various derivatives may reasonably exhibit an inhibition effect on the proliferation of mesothelioma cells as demonstrated with Nle₄-D-Phe₇-α-MSH (See FIGS. 1 and 2). (Nle₄-D-Phe₇-α-MSH is also referred to as NDP-α-MSH in this specification).

Because of this ability to inhibit NF-κB activation, α-MSH and/or its derivatives may also be used to combat other NF-κB associated tumors or cancer, for example, non-Hodgkin and Hodgkin lymphoma, lymphoid neoplasms such as cutaneous lymphomas, head and neck squamous cell carcinoma, colorectal cancer, and breast cancers. (Mayo, M. W., Baldwin, A. S., Biochimica et Biophysica Acta, 1470: M55-M62, (2000)).

Preparation and purification of α-MSH and/or its derivatives may employ conventional solid-phase peptide synthesis and reversed-phased high-performance liquid-chromatography techniques. Patients who will undergo cancer treatment may receive a pharmacologically effective amount of α-MSH and/or its derivatives either through parenteral injections or oral administration. The injections, for example, can be performed intravenously, intraperitionally, or intradermally depending on the specific location targeted. Under supervision of a physician, the patient may also receive (separately or in a single cocktail) pharmacologically effective amounts of other therapeutic cancer drugs using conventional clinical protocols.

In the case of mesothelioma, patients usually die of diffuse invasion of the pleural cavities and suffocation. Local administration of α-MSH and/or its derivatives may be preferred. For example, a cannula or any other implantable device may be placed in the pleural cavity. The cannula may contain α-MSH and/or its derivatives formulated in saline. The contents are injected through the cannula and the therapeutic peptide, alone or in conjunction with other therapeutic drugs, into the pleural cavity. The concentration of α-MSH and/or its derivatives may be in the micromolar range, and may be programmed in the implantable device, for example, to inject α-MSH and/or its derivatives twice a day. The therapeutic regimen may be continuously or periodically given (e.g., for a duration of one to four weeks). Since mesothelioma only exceptionally produces distant metastases, systemic administration is usually not required except in those exceptional circumstances.

In another embodiment of the invention, α-MSH or its derivatives may be linked, fused, or associated with a recognition molecule such as an antibody or ligand that specifically recognizes the target cancer cells. The recognition molecule may be used to target the delivery of α-MSH and/or derivatives to the specific cancer cells so as to reduce potential effects of α-MSH such as anti-inflammation, on non-cancerous cells. The linking may be performed using conventional linking techniques such as UV cross-linking, peptide fusion through recombinant DNA or peptide synthesis methods. In another embodiment of the invention, a pharmacologically effective amount of (α-MSH and/or its derivatives may also be administered to a patient with cancer either systemically or locally through a gene therapy vector expressing α-MSH and/or its derivatives.

The gene therapy vector may be comprised of a tissue specific promoter such as actin, or an inducible expression promoter such as the promoter used with the tetracyline inducible system (Clontech), ecdysone inducible system (Invitrogen, Carlsbad, Calif., or Stratagene, La Jolla, Calif.) or the GeneSwith® Inducible expression system (Invitrogen) to increase the ability to control expression of α-MSH and/or its derivatives.

In addition, α-MSH and/or its derivatives can also be expressed with an internal ribosomal entry site (IRES). The IRES sequence may be placed between another therapeutic gene such as gene encoding for an anti-angiogenic protein and the gene for α-MSH and/or its derivatives. Thus, the two genes may be transcribed as a bicistronic mRNA transcript from a single promoter, and the bicistronic mRNA, in turn, may be translated simultaneously at the 5′ end and at the IRES sequence. Because both the peptides from the therapeutic gene and α-MSH and/or its derivatives are produced from a single transcript, it is more likely that a single cell will express both proteins, thus co-localizing the effect of the two proteins. IRES sequences and vectors can be commercially obtained, for example, from Clontech Laboratories, Palo Alto, Calif. (pIRES. cat#6028-1).

Furthermore, stringing multiple genes for α-MSH and/or its derivatives using multiple IRES sequences may increase the production of α-MSH and/or its derivatives. A secretion signal peptide cloned upstream of the gene for α-MSH and/or its derivatives may also transport α-MSH and/or its derivatives to the extracellular environment where they are needed. Examples of such secretion peptide signal include the signal peptides for epidermal growth factor, basic fibroblast growth factors, or interleukin-6.

Preparation and purification of gene sequences that express α-MSH and/or its derivatives may use, among other techniques, conventional oligonucleotide synthesis techniques. Complementary oligonucleotides can be made and annealed to form double stranded DNA molecules capable of being cloned. Additional sequences representing appropriate restriction enzyme sites may be engineered at the ends of each oligonucleotide. Preferably, the oligonucleotide sequence downstream of the α-MSH sequences includes a stop codon (TTAG).

In addition, using polymerase chain reaction, a fragment corresponding to the signal peptide of IL-6 cDNA, nucleotides 33 to 120 (Genbank Accession No. J03783), may be synthesized and cloned into a vector such as pBluescript KS (Stratagene, San Diego, Calif.). Similarly, promoter regions for IL-6, NF-α, actin, or any other appropriate promoter may also be synthesized using oligonucleotides with appropriate matching restriction enzyme sites and cloned upstream of the pBluescript carrying signal sequence. Using standard restriction enzyme digestion and DNA ligation, α-MSH or its derivatives sequences may be ligated to the signal sequence and the promoter.

If an internal ribosomal entry site (IRES) sequence is desired, the oligonucleotides sequence above may include such a sequence or it can be incorporated into PCR primers and linked by conventional PCR techniques. Alternatively, the α-MSH and/or its derivatives may be cloned into the pIRES vector from Clontech Laboratories. Multiple α-MSH and/or its derivatives may be constructed with multiple IRES sequences if so desired. An effective amount of the expression plasmid containing these constructs and the therapeutic gene of interest can be directly injected or introduced into patients using non-viral vectors such as liposomes, electroporation, or using a gene gun.

Alternatively, the α-MSH and/or its derivatives constructs can be inserted into appropriate replication deficient retroviral, lentiviral, adenoviral, or adenovirus-associated-viral vectors using standard restriction enzyme and ligation techniques, blunt end cloning, or PCR techniques. Packaging cell lines using helper viruses may then package the vector DNA into viral particles for use in gene therapy.

Titer of the recombinant virus may first be determined, and the appropriate amount of viral particles may be introduced into the patients or hosts. It is understood that the viral vector may already contain a therapeutic gene or nucleic acid in addition to α-MSH and/or its derivatives.

Once the recombinant virus is introduced into cells, the cells may express α-MSH and/or its derivatives, which in turn, inhibits NF-α. The NF-α inhibition by expressing α-MSH in cells has been reported in Ichiyama, et. al., “Autocrine α-Melanocyte-Stimulating Hormone Inhibits NF-α Activation in Human Glioma,” J. Neurosci. Res. 58:684-689 (1999).

The following examples demonstrate the ability and application of α-MSH and its derivatives to combat cancer, in particular, mesotheioma proliferation. Methods in microbiology, molecular biology, and cell culture used but not explicitly described in this disclosure have already been amply reported in the scientific literature.

### EXAMPLE I

### Establishment of Seven (7) Mesothelioma Cell Lines

Various mesothelioma cell lines were established from specimens obtained from pleural effusions of patients with established pleural malignant mesothelioma. Cell cultures were performed according to standard methods. Briefly, pleural effusions were centrifuged and the cell pellets were transferred into 25 cm² tissue culture flasks. The medium consisted of RPMI 1640 supplemented with 10% fetal bovine serum, 2 mM glutamine, 10 mM HEPES buffer, 50 U/ml penicillin, and 50 μg/ml streptomycin. Cultures were maintained in humidified atmosphere of 5% CO₂ at 37° C. and examined daily. When cells were confluent, a trypsin-EDTA mixture in PBS was used to detach the cells, and the cultures were used between passages 5 and 20.

Commercially available monoclonal antibodies raised against calretinin, cytokeratin, carcino-embryonic antigen (CEA), and vimentin were then used to characterize and confirm the cells as being mesotheliomas. Based on immunocytochemistry studies, seven cell lines established had the immunocytological characteristic of mesothelial cells, i.e. co-expression of cytokeratin and vimentin and negative reactivity with antibody to CEA. Five of the tumors were epitheliomorphic and two were spindle-shaped biphasic. Three of the cell-line specimens were also examined by electron microscopy analysis for detection of characteristic microvilli and intermediate filaments, and were confirmed as mesothelioma cells.

### EXAMPLE II

### Inhibition of Mesothelioma by α-MSH

This example illustrates the inhibitory effect of α-MSH and/or its derivatives on mesothelioma cell proliferation.

1. Cell-Growth Inhibition Assay

Mesothelioma cells were counted and dispensed into eight, 96-well tissue-culture plates (Costar, Cambridge, Mass.) at a concentration of 2,000 cells/well in 100 μl of culture medium. Following a 24-hour incubation at 37° C., 5% CO₂ to allow the cells to adhere, 100 μl of culture medium containing [Nle₄-D-Phe₇]-α-MSH (kindly provided by Dr. Renato Longhi, CNR, Milano, Italy) at the final concentrations of 10⁻⁶, 10⁻⁵, 10⁻⁴M were dispensed into wells (6 replicates for each concentration). Control wells received an equal volume of medium alone. [Nle₄-D-Phe₇]-α-MSH is an analog of the natural α-MSH [amino acid 1-13] peptide in which amino acid substitutions at positions 4 and 7 provide greater chemical stability. Culture plates were then incubated for different times between 0 and 168 hours and cell proliferation was measured every 24 hours. Medium, alone or containing the same concentrations of [Nle₄-D-Phe₇]-α-MSH (from 10⁻⁶ to 10⁻⁴M), was renewed every 48 hours of incubation.

Cell proliferation was determined by a colorimetric assay using MTT [3(4,5-dimethylthiazol-2yl)2,5-diphenyltetrazolium bromide], a tetrazolium salt which is reduced to a colored formazan product by reducing enzymes present only in metabolically active cells. (See Alley et al., “Feasibility of drug screening with panel of human tumor cell lines using a microculture tetrazolium assay” Cancer Res. 48: 589-601, 1988.) Thus, metabolically active cells such as cells undergoing division will produce more formazan, which can be detected by a spectrophotometer.

Briefly, twenty-five μls of MTT solution were added to culture wells containing cells as described above. Plates were incubated at 37° C. for 3 hours. After incubation, the culture medium was removed by careful aspiration and replaced with 200 μl of DMSO to solubilize formazan. Formazan solubilization was completed by using a plate shaker for 10 min. Absorption of each well was measured using a spectrophotometer at 540 nm. The effect of α-MSH on cell proliferation was determined as percent OD difference in α-MSH treated wells relative to control wells. The cell proliferation assays were repeated in at least three separate experiments on the same cell line.

2. Effect of α-MSH on Growth of Mesothelioma Cell Lines

The mesothelioma cell lines exhibited a large variability in their sensitivity to α-MSH. Growth inhibition ranged between 10% and 40%. However, sensitivity of each cell line was consistent across separate experiments suggesting that sensitivity to α-MSH is an intrinsic characteristic of each tumor cell line.

In particular, the inhibitory effect of α-MSH occurred at the beginning of the plateau phase of cell growth in untreated cells (96 h) and was maintained thereafter until 168 h. (FIG. 1). There was no difference in cell proliferation of control and α-MSH-treated wells in earlier intervals between 24 and 72 h (not shown). Furthermore, the inhibitory effect of α-MSH was also dose-dependent. These inhibitory effects occurred over a wide range of concentrations and were significant for certain but not all cell lines with concentrations from 10⁻⁵ to 10⁻⁴M, the inhibitory effect being most significant and consistent at a concentration of 10⁻⁴M. (FIG. 2).

### EXAMPLE III

### Activity Against Selected Receptors

To provide further evidence of α-MSH activity in mesothelioma cells, immunohistochemistry using antibodies toward various melanocortin receptors has been performed to confirm the presence of melanocortin receptors in mesothelioma cells. Antibodies against the various melanocortin receptors, e.g., MC1R, MC2R, MC3R, MC4R, and MC5R were purchased from Santa Cruz Biotechnology, Inc., Santa Cruz, Calif. All the cell lines expressed the MC1R receptor. No other receptor subtype expression was detected.

### EXAMPLE IV

### Mechanism of Action

α-MSH or its derivatives inhibit the activation of NF-κB, which is associated with tumorigenesis. NF-κB factors are transcription factors consisting of dimers from the Rel family of proteins. There are five members of the NF-κB family: p50/p105 (NF-κB1), p52/p100 (NF-κB2), c-Rel, RelB, and p65 (RelA). NF-κB may be activated in the cytoplasm by phosphorylation of its inhibitor protein IκB. Proteolytic degradation of IκB also causes translocation of NF-κB to the nucleus where it binds to DNA.

NF-κB is involved in the activation of a number of genes including cytokines (such as TNF-α, IL-6, and other cytokines), growth factors, adhesion molecules, and nitric oxide synthase (NOS) as well as proto-oncogenes, such as H-ras, involved in cell proliferation and tumorigenesis (Jo H et al., “NFκB is required for H-ras oncogene induced abnormal cell proliferation and tumorigenesis” Oncogene 19:841-9, 2000).

Experiments in the monocytic cell line U937 have shown that α-MSH downregulates NF-κB activation induced by TNF-α, endotoxin, ceramide, and okadaic acid. (Manna, S. K. and Aggarwal, B. B., “α-Melanocyte-stimulating hormone inhibits the nuclear transcription factor NF-κ-B activation induced by various inflammatory agents” J. Immunol. 161, 2873-2880, (1998)). Suppression of NF-κB is mediated through inhibition of IκBα degradation. (Ichiyama, T. et al., “α-Melanocyte-stimulating hormone inhibits NF-κB activation and IκBα degradation in human glioma cells and in experimental brain inflammation” Experimental. Neural. 157, 359-365 (1999)). NF-κB's role in the development of cancer and metastasis has been described in Mayo, M. W, Baldwin, A. S., Biochimica et Biophysics Acta, 1470: M55-M62, 2000.

NF-κB is activated by certain viral transforming proteins and, in some cases is required for virus-induced transformation. Consistent with NF-κB's involvement in transformation and tumorigenesis, many human solid tumor cell lines display increased nuclear levels and/or increased NF-κB-dependent reporter activity relative to non-transformed control cell lines. For example, the classic form of NF-κB (p50-p65) is activated in breast cancer cell lines and in some breast tumors. See Sovak, M., et al., J. Clin. Invest. 100:2952-2960 (1997). Furthermore, inhibition of NF-κB in head and neck squamous cell carcinoma reduced cell survival and tumor growth. Duffey, D., et al., Cancer Res. 59: 3468-3474 (1999).

More specifically, NF-κB may also be involved in development of mesothelioma. For example, crocidolite asbestos causes prolonged, dose-related transcriptional activation of NF-κB-dependent genes. Asbestos is an established genotoxic agent that induces DNA damage, gene transcription, and protein expression important in developing malignancies such as bronchogenic carcinoma and malignant mesothelioma. It has been proposed that mesothelioma mortality can be taken as an index of past exposure to asbestos in the population. See Peto J, Hodgson J T, Matthews F E, “Jones J R: Continuing increase in mesothelioma mortality in Britain,” Lancet 1995, 345:535-539.

There are a number of autocrine and paracrine pathways involved in the proliferation of mesothelioma, and the majority of the cell lines produce significant amounts of cytokines and growth factors, including granulocyte-macrophage colony-stimulating factor, platelet-derived growth factor (PDGF), insulin-like growth factor (IGF), and interleukin-6 (IL-6). Thus, the inhibition of mesothelioma proliferation, as shown and described in Example II, may be due to inhibition of NF-κB by α-MSH and/or its derivatives. The targeting of these growth pathways, by α-MSH inhibition of NF-κB activation, is a novel approach in combating mesothelioma and cancer in general. Thus, α-MSH inhibition of NF-κB could be beneficial in treatment of tumors.

DNA-binding experiments show that α-MSH inhibits the activation and binding of NF-κB to its DNA binding site, but only in dividing cells, and not resting cells. To determine the level of NF-κB activity, nuclear extracts were prepared from 20×10⁶ U1, cells (2×10⁵/ml in complete medium) stimulated for four hours with TNF-a (20 ng/ml) in the presence or absence of 10⁻⁵M α-MSH (11-13) (KPV (SEQ. ID. NO. 1)). Cells were washed once with cold PBS, and twice with buffer A (10 mM Hepes pH 7.9, 1.5 mM MgCl₂, 10 mM KCl, 0.5 mM PMSF and 0.5 mM DTT), centrifuged, and incubated for ten minutes on ice in buffer A plus 0.1% NP-40. Afterwards, the supernatants were removed, and the nuclear pellets were resuspended in 15 μl of buffer C (20 mM Hepes pH 7.9, 1.5 mM MgCl₂, 0.42 M KCl, 0.2 mM EDTA, 25% glycerol, 0.5 mM PMSF, and 0.5 mM DTT), incubated for 15 minutes on ice, mixed, and then centrifuged. The supernatants were diluted with 75 μl of modified buffer D (20 mM Hepes, pH 7.9, 0.05 mM KCl, 0.2 mM EDTA, 20% glycerol, 0.5 mM PMSF, and 0.5 mM DTT) and stored at −80° C. The binding reaction was carried out for fifteen minutes at room temperature with 10 μl of nuclear extract protein and 0.5 ng of ³²P-labelled NF-κB (30,000 cpm/μl) or AP1 consensus in buffer A (12 mM Tris-HCl pH 7.8, 60 mM KCl, 0.2 mM EDTA, 0.3 mM DTT), plus 10% glycerol, 2 μl/ml bovine serum albumin and 1 μl/ml single stranded DNA (Pharmacia Biotech). The oligonucleotides for NF-κB used in these studies were: +GAT CCA AGG GGA CTT TCC GCT GGG GAC TTT CCA TG (SEQ. ID. NO. 8) and −GAT CCA TGG AAA GTC CCC AGC GGA AAG TCC CCT TG (SEQ. ID. NO. 9). Each oligonucleotide was annealed to its complementary strand and end-labeled with ³²P-γ-ATP using polynucleotide kinase. For the determination of specific bands, nuclear extracts were first incubated with 100 fold excess unlabeled probe for five minutes, before incubation with a labeled probe. The mixtures were then run on 5%(30:1) acrylamide gel in 1×TBE. The gels were dried and autoradiographed.

FIG. 4 shows that TNF-α greatly enhanced NF-κB binding activity, but the co-incubation of α-MSH (11-13) (KPV (SEQ. ID. NO. 1)) at 10⁻⁵M significantly reduced NF-κB activation. In resting cells, however, α-MSH (11-13) (KPV (SEQ. ID. NO. 1)) did not alter NF-κB activation. Thus, α-MSH inhibits NF-κB activation in dividing cells, and may be used for treatment of cancer in which activation of NF-κB is prominent.

Similar DNA-binding assays may be performed on NF-κB activation in malignant mesothelioma relative to normal mesothelium that would further prove α-MSH inhibition of NF-κB in cancer cells.

### EXAMPLE V

### Derivatives

As used herein, the term “α-MSH and/or its derivatives” means any molecule derived from α-MSH (SEQ. ID. NO. 4) by deletion, substitution, modification of the amino acids in α-MSH, or, linking, coupling, fusion, or association with other peptides or molecules. The term also means any dimer (e.g. homodimer or heterodimer) of the various molecules derived from α-MSH (SEQ. ID. NO. 4).

The above assays and experiments in the Examples above were performed using α-MSH derivatives, [Nled₄-D-Phe₇]-α-MSH and KPV. [Nle₄-D-Phe₇]-α-MSH is preferred, for example, because of its greater stability compared to α-MSH. In addition, [Nle₄-D-Phe₇]-α-MSH also greatly increases the biological activity of the α-MSH peptide. For example [Nle₄-D-Phe₇]-α-MSH has biological activity on melanocytes and melanoma cells as with α-MSH, but is approximately ten times more potent than the parent peptide in reducing fever.

Further stabilization of the α-MSH sequence by substituting D-amino acid forms for L-forms of the amino acids may also be accomplished. For example, D-amino acid substitution may include AC-[D-K11]-α-MSH 11-13-NH₂, which has the same general potency as the L-form of the tripeptide α-MSH (11-13) (SEQ. ID. NO. 1). See e.g. Holdeman, M., et. al., Antipyretic Activity of a Potent α-MSH Analog, Peptides 6, 273-5 (1985). Deeter, L. B., et. al., Antipyretic Properties of Centrally Administered α-MSH Fragments in the Rabbit, Peptides 9, 1285-8 (1989). Hiltz, M. E., Anti-inflammatory Activity of α-MSH (11-13) Analogs: Influences of Alterations in Stereochemistry, Peptides 12, 767-71 (1991). The KPV tri-peptide is preferred because it is a smaller molecule, which is more likely to increase access to certain parts of the body (e.g., the blood-brain barrier in the central nervous system).

Although [Nle₄-D-Phe₇]-α-MSH or KPV may be preferred, it should be understood that the parent molecule, α-MSH, and other biologically functional equivalents of α-MSH may also be used to combat cancer. For example, various α-MSH derivatives such as N-terminal truncations of α-MSH (e.g., the 10-13 sequence of α-MSH (SEQ. ID. NO. 5), the 9-13 sequence of α-MSH (SEQ. ID. NO. 6), the 8-13 sequence of α-MSH (SEQ. ID. NO. 7), and any other polypeptides having a C-terminal KPV may also be used. Furthermore, chemical modifications such as N-acetylation and/or C-amidation may be used to stabilized α-MSH or peptides derived from α-MSH.

Biologically functional equivalents can also be obtained by substitution of amino acids having similar hydropathic values. Thus, for example, isoleucine and leucine, which have a hydropathic index +4.5 and +3.8, respectively, can be substituted for valine, which has a hydropathic index of +4.2, and still obtain a protein having like biological activity. Alternatively, at the other end of the scale, lysine (−3.9) can be substituted for arginine (−4.5), and so on. In general, it is believed that amino acids can be successfully substituted where such amino acids have a hydropathic score of within about +/−1 hydropathic index unit of the replaced amino acid.

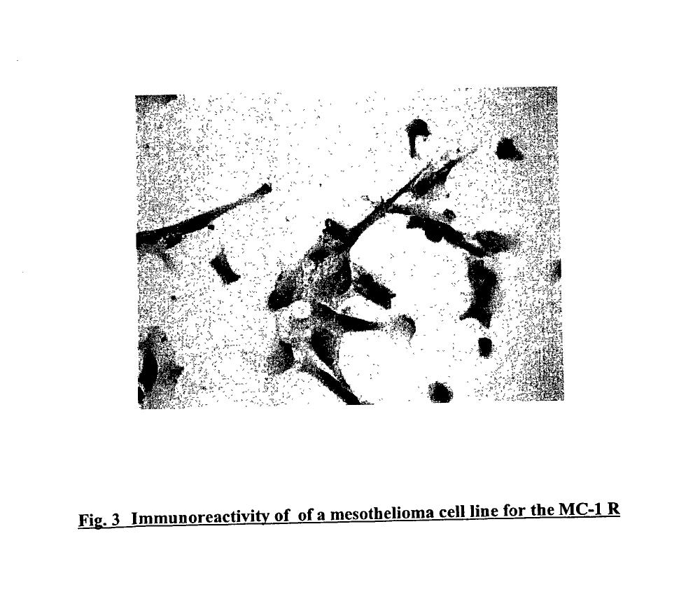

Furthermore, these modified analogs of α-MSH and/or its derivatives can also form dimers as exemplified by the KPV dimer in FIG. 3.

### EXAMPLE VI

### Use in Mesothelioma

A patient is diagnosed as having cancer such as malignant mesothelioma or other NF-κB related cancer. The patient may undergo a conventional therapeutic regimen including chemotherapy, surgery, or radiotherapy at the direction of the appropriate medical practitioner, an oncologist. For example, in addition to the conventional therapeutic regimen, the patient may be given a pharmacologically effective amount of α-MSH and or its derivatives systemically using injection into the circulatory system at intervals directed by the appropriate medical practitioner. Alternatively, α-MSH and/or its derivatives may be administered locally by injection or using an implantable device such as a cannula that secretes the peptides into a body cavity. Further examples of administration may include administering α-MSH and/or its derivatives using gene therapy protocols.

From the foregoing, it will be appreciated that specific embodiments of the invention have been described herein for purposes of illustration, but that various modifications may be made without deviating from the spirit and scope of the invention. The preceding Examples are intended only as examples and are not intended to limit the invention. It is understood that modifying the examples above does not depart from the spirit of the invention. It is further understood that each example may be applied on its own or in combination with other examples.

1. The use of α-MSH or a derivative of α-MSH, or both, to combat cancer.

2. The use in claim 1 wherein the derivative of α-MSH is a polypeptide comprising a C-terminal KPV.

3. The use in claim 1 wherein the derivative of α-MSH is a D-amino acid substituted form of α-MSH where any single amino acid or any combination of amino acids may be of the D configuration.

4. The use in claim 3 wherein the D-amino acid substituted form of α-MSH is [Nle₄-D-Phe₇]-α-MSH.

5. The use in claim 1 wherein the derivative of α-MSH is a dimer comprising polypeptides having C-terminal KPV.

6. The use in claim 1 wherein the cancer is mesothelioma.

7. The use in claim 1 wherein the cancer is selected from group consisting of hodgkin lymphoma, non-hodgkin lymphoma, squamous cell carcinoma, breast cancer, and colorectal cancer.

8. The use in claim 1 wherein the α-MSH or the derivative of α-MSH, or both, is delivered locally using a cannula implanted in a body cavity.

9. The use in claim 1 wherein the α-MSH or the derivative of α-MSH, or both, is delivered using oral, parenteral or a gene therapy vector comprising a nucleic acid sequence encoding for α-MSH or the derivative of α-MSH.

10. The use in claim 10 wherein the gene-therapy vector further comprises a tissue specific promoter.

11. The use in claim 10 wherein the gene-therapy vector further comprises an inducible expression vector.

12. The use in claim 10 wherein the gene therapy vector further comprises an internal ribosomal entry site and a second nucleic acid sequence encoding for an anti-angiogenic gene.

13. The use in claim 10 wherein the α-MSH or the derivative of α-MSH, or both, is linked to a recognition molecule that binds to a cancer cells.

14. The use in claim 1 wherein the α-MSH or the derivative of α-MSH, or both, inhibits the proliferation of cancer cells.

15. A use for α-MSH and/or its derivatives in inhibiting cancer cell proliferation comprising the step of:
administering a pharmacologically effective amount of α-MSH or a derivative of α-MSH, or both, to a patient with cancer.

16. The use in claim 16 wherein the α-MSH or the derivative of α-MSH, or both, may be administered locally, directly, or parenterally to cancerous cells.

17. The use in claim 17 wherein the α-MSH or the derivative of α-MSH, or both, is administered locally using a cannula implanted in a body cavity of the patient.

18. A therapeutic molecule comprising α-MSH or the derivative of α-MSH, or both, linked to a recognition molecule that recognizes cancer cells.


SEQUENCE LISTING (9 sequences)

SEQ ID NO: 1 - protein, 3 aa, Artificial Sequence
  Designed polypeptide with anti-inflammatory, anti-microbial, anti-fungal, anti-viral, and anti-cancer properties.
KPV

SEQ ID NO: 2 - protein, 7 aa, Artificial Sequence
  Designed polypeptide with anti-inflammatory, anti-microbial, anti-fungal, and anti-viral properties.
MEHFRWG

SEQ ID NO: 3 - protein, 8 aa, Artificial Sequence
  Designed polypeptide with anti-inflammatory, anti-microbial, anti-fungal, anti-viral, and anti-cancer properties.
HFRWGKPV

SEQ ID NO: 4 - protein, 13 aa, Homo sapiens
SYSMEHFRWGKPV

SEQ ID NO: 5 - protein, 4 aa, Artificial Sequence
  Designed polypeptide with anti-inflammatory, anti-microbial, anti-fungal, anti-viral, and anti-cancer properties.
GKPV

SEQ ID NO: 6 - protein, 5 aa, Artificial Sequence
  C-terminal sequences of alpha-MSH
WGKPV

SEQ ID NO: 7 - protein, 6 aa, Artificial Sequence
  Designed polypeptide with anti-inflammatory, anti-microbial, anti-fungal, and anti-viral properties.
RWGKPV

SEQ ID NO: 8 - DNA, 35 bp, Artificial Sequence
  NF-kappa B DNA binding site, positive strand
gatccaaggggactttccgctggggactttccatg

SEQ ID NO: 9 - DNA, 35 bp, Artificial Sequence
  NF-kappa B DNA binding site, negative strand
gatccatggaaagtccccagcggaaagtccccttg